(19) United States
(12) Patent Application Publication
(10) Pub. No.: US 2025/0285077 A1
(43) Pub. Date: Sep. 11, 2025

(54) INVENTORY MANAGEMENT SYSTEM

(71) Applicant: VETSNAP CORP., Thousand Oaks, CA (US)

(72) Inventors: Steven Patrick ELLIOTT, Thousand Oaks, CA (US); Yangyang SHI, Thousand Oaks, CA (US)

(21) Appl. No.: 19/076,698

(22) Filed: Mar. 11, 2025

Related U.S. Application Data
(60) Provisional application No. 63/563,767, filed on Mar. 11, 2024.
(60) Provisional application No. 63/639,962, filed on Apr. 29, 2024.

(51) Int. Cl. G06Q 10/087; G06Q 30/0601; G16H 10/60; G16H 20/10

(52) U.S. Cl. CPC G06Q 10/087; G06Q 30/0607; G16H 10/60; G16H 20/10

(57) ABSTRACT

Technologies are disclosed for a hardware and software integrated inventory management system. A system receives a request from a client device of a user to access an inventory storage device storing one or more controlled substances. The system causes the inventory storage device to provide access to the client device. The system receives a hardware log generated by the inventory storage device, which provides one or more records relating to the access of the inventory storage device by the user. Each record providing information relating to one of the one or more controlled substances removed from the inventory storage device. An entry for a controlled substance log maintained by the system is generated for each record, and the system updates the controlled substance log with the generated entries.

### CROSS REFERENCE TO RELATED APPLICATIONS

The present application claims priority to U.S. Provisional Application No. 63/563,797, filed on Mar. 11, 2024 and to U.S. Provisional Application No. 63/639,962, filed Apr. 29, 2024.

### FIELD

Embodiments disclosed herein generally relate to practice management and inventory systems, and more specifically, to technologies for managing a hardware-integrated inventory management system.

### BACKGROUND

In the medical industry, a practice (e.g., a clinic, hospital, pharmacy, etc.) must, by law, record and track usage of controlled substances in patients. Generally, controlled substances are chemicals, pharmaceutical agents, and the like that have been identified by a governmental body as having a potential for abuse and dependency. For example, the United States Drug Enforcement Agency categorizes and regulates controlled substances based on medicinal use, potential for abuse, and safety or dependence liability. The practice generally maintains the controlled substance records in a controlled substance log, which is subject to various requirements by regulatory agencies. Current technologies allow a veterinary practice to electronically maintain controlled substance inventories. For example, cloud-based solutions exist that enable practitioners at a given veterinary clinic to manage the controlled substance log remotely, such as from different locations within the veterinary practice via different computing devices.

Tracking usage of controlled substances typically involves two sources: a controlled substance log and a practice information management system (PIMS). An individual at the practice (e.g., a veterinary doctor, assistant, or other staff) must input usage information in the controlled substance log as the usage occurs throughout the day. The PIMS maintains patient medical records, which includes itemized controlled drug substance usage for that patient entered generally for accounting purposes.

### SUMMARY

One embodiment presented herein discloses a method. The method generally includes receiving, by a platform application executing on one or more processors, a request from a client device of a user to access an inventory storage device storing one or more controlled substances. The method also generally includes causing the inventory storage device to provide access to the client device. The method also generally includes receiving a hardware log generated by the inventory storage device. The hardware log provides one or more records relating to the access of the inventory storage device by the user, and each record provides information relating to one of the one or more controlled substances removed from the inventory storage device. The method also generally includes generating, for each of the one or more records, an entry for a controlled substance log maintained by the platform application. The controlled substance log is updated with the generated entry for each of the one or more records.

Another embodiment presented herein discloses a system having one or more processors and a memory storing instructions, which when executed by the one or more processors, causes the system to perform an operation. The operation generally includes receiving, by a platform application, a request from a client device of a user to access an inventory storage device storing one or more controlled substances. The operation also generally includes causing the inventory storage device to provide access to the client device. The operation also generally includes receiving a hardware log generated by the inventory storage device. The hardware log provides one or more records relating to the access of the inventory storage device by the user, and each record provides information relating to one of the one or more controlled substances removed from the inventory storage device. The operation also generally includes generating, for each of the one or more records, an entry for a controlled substance log maintained by the platform application. The controlled substance log is updated with the generated entry for each of the one or more records.

Yet another embodiment presented herein discloses one or more non-transitory computer-readable storage media storing instructions, which when executed on one or more processors, causes a system to perform an operation. The operation generally includes receiving, by a platform application, a request from a client device of a user to access an inventory storage device storing one or more controlled substances. The operation also generally includes causing the inventory storage device to provide access to the client device. The operation also generally includes receiving a hardware log generated by the inventory storage device. The hardware log provides one or more records relating to the access of the inventory storage device by the user, and each record provides information relating to one of the one or more controlled substances removed from the inventory storage device. The operation also generally includes generating, for each of the one or more records, an entry for a controlled substance log maintained by the platform application. The controlled substance log is updated with the generated entry for each of the one or more records.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a simplified conceptual diagram of an example computing environment in which a hardware-integrated inventory management system is deployed, according to an embodiment;

FIG. 2 is a simplified block diagram of the platform server described relative to FIG. 1, according to an embodiment;

FIG. 3 is a simplified block diagram of the client device described relative to FIG. 1, according to an embodiment;

FIG. 4 is a simplified block diagram illustrating components of the inventory storage device described relative to FIG. 1, according to an embodiment;

FIG. 5 is a conceptual diagram illustrating a user interface view of the inventory management application of FIG. 1 for managing substance inventory of a physical storage device, according to an embodiment;

FIG. 6 is a sequence diagram for performing a hardware-based inventory transaction within the inventory management system of FIG. 1, according to an embodiment;

FIG. 7 is a sequence diagram for performing a hardware-based inventory transaction within the inventory management system of FIG. 1, in which the inventory storage device interfaces with a third-party provider, according to an embodiment;

FIG. 8 is a flow diagram for generating multiple distinct controlled substance entries from a single user interface view, according to an embodiment; and

FIG. 9 is a conceptual diagram illustrating a user interface view of the inventory management application of FIG. 1 for generating multiple distinct controlled substance entries, according to an embodiment.

### DETAILED DESCRIPTION

Current approaches to tracking medical substance inventory, such as inventory of controlled substances that are subject to stringent regulatory requirements, are insufficient. Generally, medical practices (e.g., medical clinics, veterinary hospitals, etc.) are busy practices in which multiple staff members require access to controlled substances to treat patients. Typically, the substances are stored in lockers (or other containers) that are controlled through some security mechanism (a lock, numpad, access panel, etc.) and log. In such practices, inventory counts and controlled substance logs are audited on a regular basis but are often filled with discrepancies in need of reconciliation. Further, audit trails often reveal inconsistencies as access codes can be shared frequently by medical staff members, so accurately tracking inventory transactions (and individuals conducting the transactions) has some difficulties.

Embodiments presented herein disclose improvements in hardware-based inventory management technologies. More particularly, the disclosed technologies provide hardware-and software-integrated system for user-permissioned access control and logging in managing and tracking medical substances, such as controlled substances. Embodiments provide an inventory management system platform that integrates with a practice inventory management system (PIMS) of a medical or veterinary practice. The platform may serve as one source of controlled usage data, providing a digital controlled substance usage log (also referred to herein as a “digital logbook”) that the veterinary practice can use (e.g., via a client device executing a platform application thereon) to enter standardized usage data throughout the day while treating patients. The platform provides a mobile application that can be executed on a client device that allows users (e.g., employees within a medical or veterinary practice) to access and update substance inventories.

The platform and digital logbook may be integrated with one or more hardware inventory storage devices (e.g., safes, lockers, storage boxes, smart lockers, etc.) used to store the medical substances to achieve more accurate tracking of the substances. The platform may include an application programming interface (API) that is capable of communicating with communication interfaces of the inventory storage device, advantageously allowing for the platform to track items that are taken in and out of the storage device through the digital logbook.

Further, the disclosed technologies provide techniques for multidrug logging for controlled substances within a medical practice within a single user interface view. The disclosed technology provides a graphical user interface (GUI) that allows a user to log controlled substance usage for any medication being used and select and option to include additional medications to the controlled substance log. The GUI provides the option to associate mandatory information (e.g., mandatory for regulatory compliance, such as container name, quantity, drug name, etc.) with each of the controlled substances entered during the process, thereby seamlessly generating multiple individual controlled substance log entries.

Advantageously, the technologies disclosed herein provide a secure approach for managing access to controlled substances. By integrating hardware storage lockers with a software-based inventory management system, activity by users (e.g., medical staff, veterinary staff, hospital administrators, etc.) can be efficiently tracked.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

Note, the following disclosure uses controlled substances as a reference example for items that may be controlled and precisely tracked using hardware-integrated inventory management systems. However, one of skill in the art will recognize that the broader inventive concept can be adapted to a variety of inventory-based transactions, such as the dispensing or administering of substances at a medical practice.

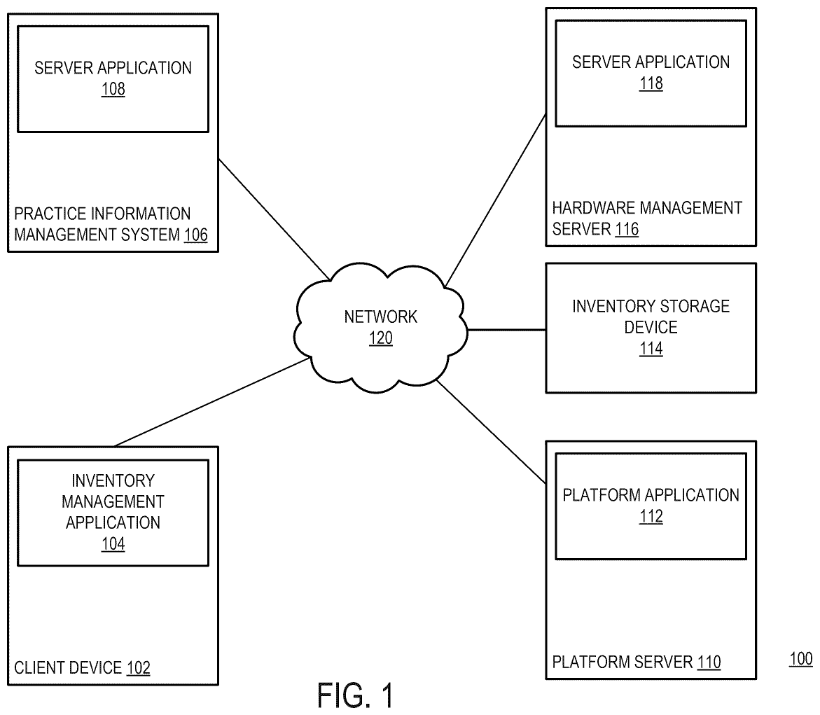

Referring now to FIG. 1, a computing environment 100 in which controlled substance usage data from multiple sources is tracked across various locations within a medical practice is shown, according to an embodiment. As shown, the computing environment 100 includes a client device 102, a practice information management system (PIMS) 106, a platform server 110, one or more inventory storage devices 114, and a hardware management server 116, each interconnected via a network 120 (e.g., the Internet, a local area network, private network, etc.).

The client device 102 may be embodied as a physical computing device (e.g., a desktop computer, laptop computer, mobile device such as a mobile tablet or a smartphone, etc.) or a virtual computing instance executing in a cloud network. A user of the client device 102 may be an individual employed at a medical practice, such as a veterinary practice (e.g., a veterinary clinic, hospital, office with a pharmacy, etc.) that administers treatment to an animal patient, such as dispensing and administering medication to the patient, including controlled substances. As used herein, a controlled substance pertains to a drug or a chemical that is subject to governmental regulations such as the Controlled Substances Act (CSA) with regard to manufacture, possession, and use.

Illustratively, the client device 102 includes an inventory management application 104. The inventory management application 104 provides a drug inventory management interface enabling the user to record controlled substance usage over the course of the day. The interface may be embodied as a “digital logbook” that maintains records of usage and controlled substance inventory of the practice. For example, the user may, via the inventory management application 104, record entries of controlled substance usage into the digital logbook provided by the interface. The interface may allow the user to enter, for example, patient information (e.g., name, chart ID, owner name, treatment), drug name, quantity, physician information, approval information, and so on. Further, the inventory management application 104 is integrated with one or more PIMS, such as PIMS 106.

The PIMS 106 may be a physical computing system (e.g., a desktop computer, workstation computer, laptop computer, etc.) or a virtual computing instance executing in the cloud. The PIMS 106 can also run on the premises of the veterinary practice or a remote location. As shown, the PIMS 106 further includes a server application 108, providing known functionalities of a PIMS, including managing patient data (including maintaining records of controlled substance usage per patient), invoicing, inpatient care workflows, reporting workflows, and so on. In an embodiment, the inventory management application 104 is incorporated into an inventory management platform that includes the platform server 110. In other embodiments, one or a combination of the functions performed by the inventory management application 104 and platform application 112 can be performed by a single application.

In an embodiment, the platform server 110 stores and maintains standardized PIMS data for processing by the inventory management application 104. To do so, the platform server 110 includes a platform application 112 executing thereon that performs such functions. The platform application 112 may provide an API to the inventory management application 104 to enable communication (e.g., transmission of PIMS data, local controlled usage log data, accountability audit report generation, etc.) between the applications 104 and 112. In other embodiments, the platform application 112 also maintains and stores usage data provided by multiple client devices 102 each executing an instance of the inventory management application 104 (e.g., in the event that a client device 102 is a mobile device such as a tablet, and the inventory management application 104 is operated by multiple users). In other further embodiments, the platform server 110 manages data recorded in the digital logbook of the inventory management application 104 and tracks inventory and usage of controlled substances during normal operation of the medical practice. The platform application 112 may use APIs provided by the server application 116 to communicate with the PIMS 106.

As stated, controlled substance usage data may be recorded and maintained by the various components of the computing environment 100. For example, the inventory management application 104 maintains a controlled substance usage log in a digital logbook. Further, the PIMS data includes controlled substance usage information. The platform application 112 (or the inventory management application 104) may validate and reconcile controlled substance usage data of multiple sources. To ensure this accuracy in the controlled substance usage data, the platform application 112 verifies and matches usage data of drugs being tracked from each individual source. The platform application 112 uses identifiable information such as drug name, date of usage, client, or patient chart number, and other typically recorded data.

In an embodiment, the inventory management application 104 and platform application 112 may manage the inventory balances of controlled substances using container objects. A container object is a data object that is analogous to a physical container used to store up to a certain quantity of a given controlled substance at the medical practice. In addition to storing information regarding a given controlled substance, the inventory management application 104 and platform application 112 may also associate quantities of a controlled substance to one or more container objects.

In an embodiment, the inventory management application 104 may provide a graphical user interface (GUI) that enables a user to enter multiple controlled substances from a single GUI view on the device. Once complete, the inventory management application 104 may generate, from data provided by the user in the view, multiple controlled substance usage log records. Doing so advantageously reduces the amount of time taken to enter the records into the log. In an embodiment, the GUI is a web-based interface accessed by the inventory management application 104 in communication with a web server provided by the platform application 112.

The inventory storage device 114 may be embodied as any type of electronic storage device configured to securely store controlled substances on behalf of the medical practice while in compliance with government regulations for storing controlled substances. For example, the inventory storage device 114 can be a modular storage locker, a safe, a locked cabinet, an automated medication dispensing cabinet, smart locker, specific-purpose narcotic safe, etc. The inventory storage device 114 may be configured to communicate over the network 120 using network communications components.

The inventory storage device 114 may be centrally managed by the platform server 110 (e.g., via the server application 108). In some embodiments, the inventory storage device 114 may operate as an Internet-of-Things (IoT) device in communication with and managed by a cloud-based (and optionally third-party) hardware management server 116. The hardware management server 116 may provide a server application 118 that manages operations, user access, and inventory tracking operations for the device. As further described herein, the inventory storage device 114 includes logic that enables tracking and management by the platform application 112 of items being stored therein and removed therefrom, advantageously allowing for contemporaneous generation of controlled substance log entries for the medical practice in response to storage and removal events relating to the inventory storage device 114.

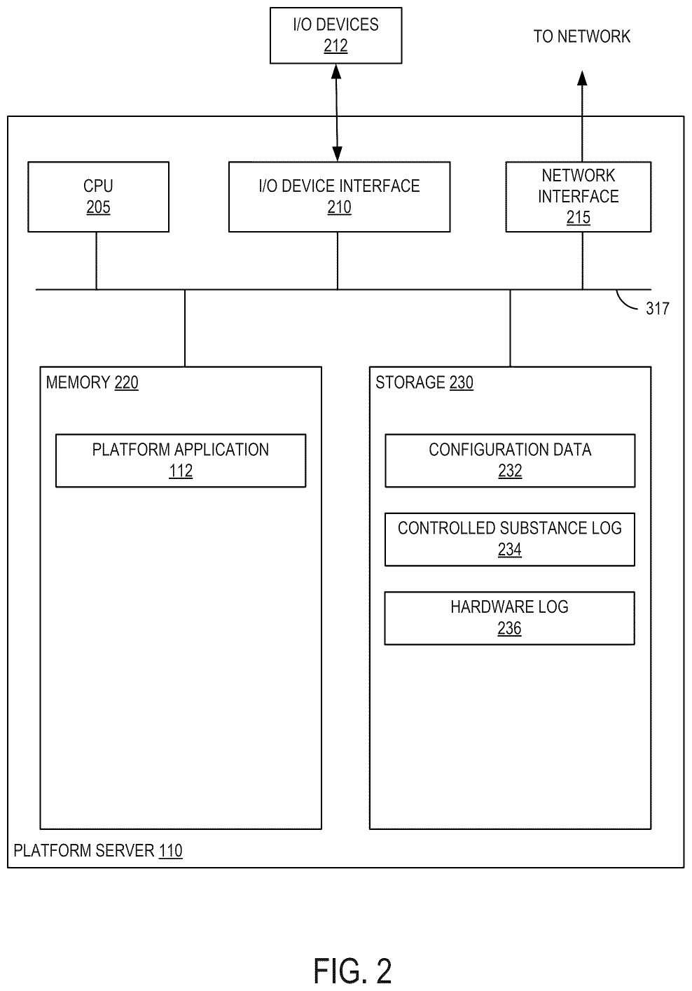

Referring now to FIG. 2, a block diagram illustrating components of the platform server 110. As shown, platform server 110 includes, without limitation, a central processing unit (CPU) 205, an input/output (I/O) device interface 210, one or more I/O devices 212, a network interface 215, a memory 220, and a storage 230, each interconnected via a hardware bus 217. Of course, the actual platform server 110 will include a variety of additional hardware components not shown. Additionally, in some embodiments, one or more of the illustrative components may be incorporated in, or otherwise form a portion of, another component.

The CPU 205 retrieves and executes programming instructions stored in the memory 220 (e.g., of the platform application 112). The CPU 205 may be embodied as one or more processors, each processor being a type capable of performing the functions described herein. For example, the CPU 205 may be embodied as a single or multi-core processor(s), a microcontroller, or other processor or processing/controlling circuit. In some embodiments, the CPU 205 may be embodied as, include, or be coupled to a field programmable gate array (FPGA), an application-specific integrated circuit (ASIC), reconfigurable hardware or hardware circuitry, or other specialized hardware to facilitate performance of the functions described herein. The hardware bus 217 is used to transmit instructions and data between the CPU 205, storage 230, network interface 215, and the memory 220. CPU 205 is included to be representative of a single CPU, multiple CPUs, a single CPU having multiple processing cores, and the like. The memory 220 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 215 may be embodied as any hardware, software, or circuitry (e.g., a network interface card) used to connect the platform server 110 over the network 120 and providing the network communication component functions described above. For example, the network interface 215 may be embodied as any communication circuit, device, or collection thereof, capable of enabling communications over the network 120 between the platform server 110 and other devices (e.g., the client device 102, inventory storage device 114, hardware management server 116, etc.). The network interface 215 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 215 may include a network interface controller (NIC, not shown), embodied as one or more add-in-boards, daughtercards, controller chips, chipsets, or other devices that may be used by the platform server 110 for network communications with remote devices. For example, the NIC may be embodied as an expansion card coupled to the I/O device interface 210 over an expansion bus such as PCI Express.

The I/O device interface 210 allows the I/O devices 212 to communicate with hardware and software components of the platform server 110. For example, the I/O device interface 210 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 210 may form a portion of a system-on-a-chip (SoC) and be incorporated, along with one or more of the CPU 205, the memory 220, and other components of the platform server 110. The I/O devices 212 may be embodied as any type of I/O device connected with or provided as a component to the platform server 110. I/O devices such as keyboards, mice, and printers may be included as I/O devices 212 (e.g., to access an administrator UI, print controlled substance transaction records, etc.).

The memory 220 includes the platform application 112, which performs the various functions described herein for managing and tracking controlled substance transactions and records for a digital logbook of a medical practice.

The storage 230 may be embodied as any type of devices configured for short-term or long-term storage of data such as, for example, memory devices and circuits, memory cards, hard disk drives (HDDs), solid-state drives (SSDs), or other data storage devices. The storage 230 may include a system partition that stores data and firmware code for the storage 230. The storage 230 may also include an operating system partition that stores data files and executables for an operating system. The storage 230 includes configuration data 232, a controlled substance log 234, and a hardware log 236. The configuration data 232 may comprise any data pertaining to the configuration of the platform application 112 and the client device-side inventory management application 104, such as network addresses and locations of the inventory storage devices 114, user access control data for the inventory storage devices 114, API and integration settings (e.g., API keys and tokens, credentials, etc.), database configurations, compliance settings, and the like. The controlled substance log 234 is a server-side instance of the digital logbook maintained by the medical practice. The hardware log 236 comprises records of interactions and events relating to access to each of the inventory storage devices 114 maintained by the medical practice. For example, the hardware log 236 may include timestamp data, accessing user data, authentication method used (e.g., RFID, numpad, biometric scan, Bluetooth, mobile application function, etc.), identifier of the inventory storage device 114 accessed, access type (e.g., open, close), duration of the storage device 114 being open, failed attempt data, etc. The hardware log 236 also includes item information such as drug identifier, dosage, container identifier, patient information, client information, accessing user identifier, quantity, weight, expiration date information, etc.

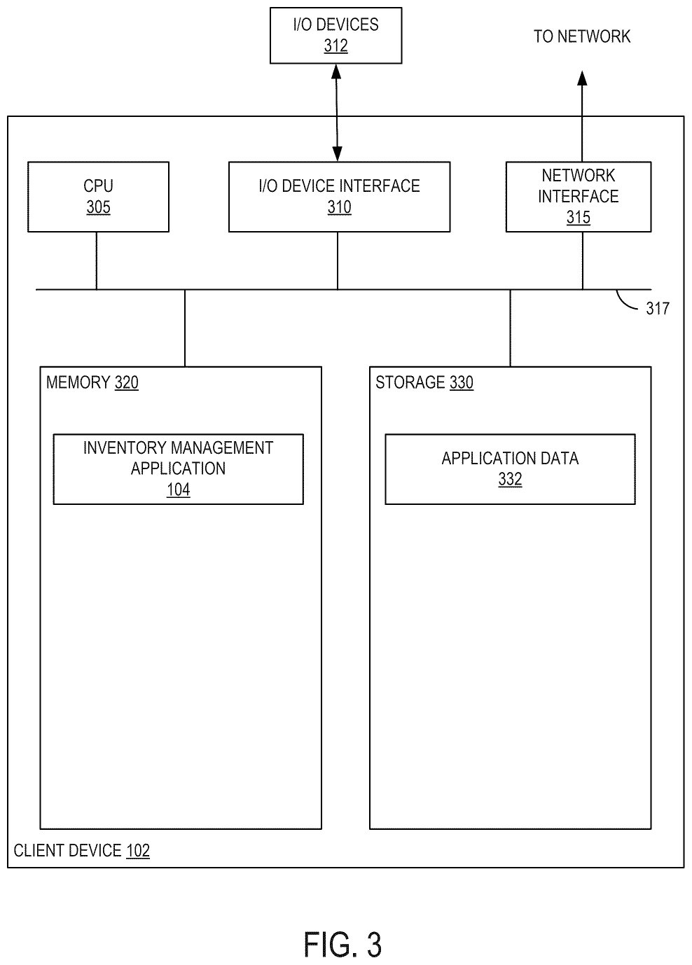

Referring now to FIG. 3, a block diagram of the client device 102 is shown. As shown, client device 102 includes, without limitation, a central processing unit (CPU) 305, an input/output (I/O) device interface 310, one or more I/O devices 312, a network interface 315, a memory 320, and a storage 330, each interconnected via a hardware bus 317. Of course, the actual client device 102 will include a variety of additional hardware components not shown. Additionally, in some embodiments, one or more of the illustrative components may be incorporated in, or otherwise form a portion of, another component. Further, each of the CPU 305, I/O interface 310, I/O devices 312, network interface 315, memory 320, and storage 330 are similar to the respective components described relative to FIG. 2 of the platform server 110.

The memory 320 includes the inventory management application 104. The storage 330 includes application data 332, which can include any data generated, used, and/or maintained by the inventory management application 104, such as configuration settings, user management data, digital logbook instances for the medical practice, user credentials, server credentials, and the like.

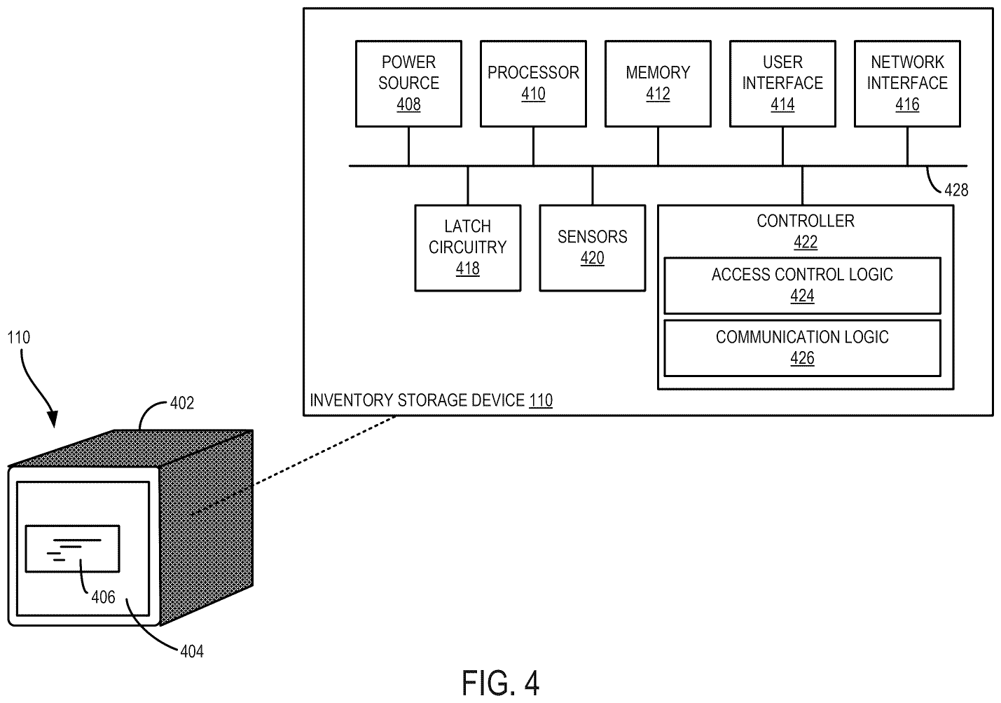

Referring now to FIG. 4, a perspective view of the exterior of the inventory storage device 110 is shown with a block diagram of components thereof. As shown, the inventory storage device 110 includes a body 402, door 402, and an access mechanism 406. The body 402 may be formed of any suitable material for ensuring security and condition of controlled substances stored within the inventory storage device 110. For example, the body 402 may be formed of reinforced steel to prevent easy break-in. The access mechanism 406 may provide electronic locks and access devices (e.g., numpad for entering a secure personal identification number (PIN), NFC reader, RFID ready, keycard reader, biometric scanner, mechanical locks, touchscreen interface, etc.) used to control access to the inventory storage device 110 via the door 402.

Illustratively, components of the inventory storage device 110 may include a power source 408, processor 410, memory 412, user interface 414, network interface 416, latch circuitry 418, sensors 420, and controller 422, which itself includes access control logic 424 and communication logic 426, each interconnected via bus 428 (used to transmit instructions and data between the components).

The power source 408 provides power and connectivity for the inventory storage device 110, e.g., using a 110V-240 AC connection to ensure continuous operation thereof. The processor 410 and memory 412 are components enabling the smart functions of the inventory storage device 110, including execution of access logging, item tracking, access control, and the like. The processor 410 and memory 412 may be embodied similarly as the CPU 205 and memory 220 of FIG. 2, respectively. The user interface 414 provides access, transaction management, and monitoring functions to a user of the inventory storage device 110. For instance, the user interface 414 may be coupled with the access mechanism 406 in prompting a user for credentials to access the inventory storage device 110. The user interface 414 may also provide a means for a user to access functions of the inventory storage device 110 through the inventory management application 104 executing on the client device 102. The network interface 416 enables communication between the inventory storage device 110 and other devices in the network 120 (e.g., the client device 102, platform server 110, hardware management server 116, etc.). The network interface 416 may be embodied similarly to the network interface 215 of FIG. 2.

The latch circuitry 410 may be embodied as a component that controls locking and unlocking of the inventory storage device. The latch circuitry 410 may interface with an actuator to engage or release the lock when access is granted. The latch circuitry 410 may operate in response to an authorization signal sent by the controller 422. Sensors 420 can include any type of sensor for providing security, tracking inventory, and controlling access for the inventory storage device 114. For example, the sensors 420 can include RFID and/or NFC readers, biometric scanners, numpads, touchscreen sensors, and the like, for access control. The sensors 420 can also include security cameras, weight sensors (e.g., scales), infrared or optical sensors, barcode scanners, and QR scanners for inventory tracking.

The controller 422 manages operations for the inventory storage device 114, such as access control and authentication, lock mechanism control, inventory management, communication, power management, and the like, via connections to the power source 408, processor 410, memory 412, user interface 414, network interface 416, latch circuitry 418, and sensors 420 over the bus 428. The access control logic 424 manages and enforces authentication and authorization rules for access to the inventory storage device 114. The access control logic may determine who has access to the inventory storage device 114 based on such authentication and authorization rules and transmit signals to unlock locking mechanisms in response to successful authentication. The communication logic 426 handles data communications between the components of the inventory storage device 114 over external networks, such as network 120. The communication logic 426 handles communication protocols (e.g., WiFi, Bluetooth, LTE, Ethernet) for data transmission, manages data synchronization between the inventory storage device 114 and the platform server 110 and hardware management server 116, manages data exchange over the bus 428, and so on.

Although FIG. 4 depicts the inventory storage device 110 as a safe locker, one of skill in the art will appreciate that the technologies disclosed herein can be adapted toward a variety of smart storage devices having network communication and software integration capabilities. Further, the inventory storage locker 110 may also include a number of components not currently shown, such as antennas, drawers, locker storage units, storage drawers and/or shelves, automatic dispensing units, and so on.

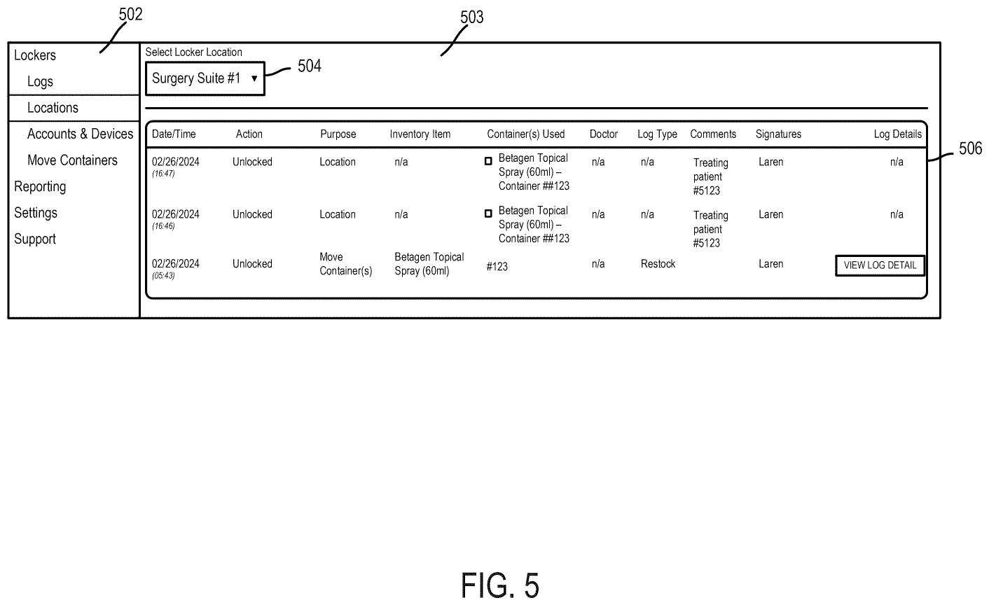

Referring now to FIG. 5, a graphical user interface (GUI) view 500 of the inventory management application 104 executing on the client device 102 is shown. The view 500 provides a management interface for one or more inventory storage devices 114 (also referred to herein as “lockers”) managed by the medical practice. The view 500 includes a left panel 502 that provides a menu of accessible items (“Lockers”, “Reporting”, “Settings”, and “Support”) and sub-items (“Logs”, “Locations”, “Accounts & Devices” and “Move Containers” for menu item “Locker”) for display on a right display panel 503. The example view 500 displays information relating to inventory storage devices 114 deployed at various locations of the medical practice (e.g., different medical facilities, different areas within a given location, etc.). For instance, the present example displays information relating to an inventory storage device 114 deployed at a “Surgery Suite #1”, as shown in the selectable dropdown box 504. The display panel 503 provides a table 506 showing information relating to the inventory storage device 114 at “Surgery Suite #1” in a row and column format, in which each row corresponds to event interactions with the inventory storage device 114. The columns list fields for each record, such as “Date/Time”, “Action”, “Purpose”, “Inventory Item”, “Container(s) Used”, “Doctor”, “Log Type”, “Comments”, “Signatures”, and “Log Details”. Each record may be entered in a hardware log associated with the inventory storage locker 114 and managed by the platform server 110 (and/or the hardware management server 116).

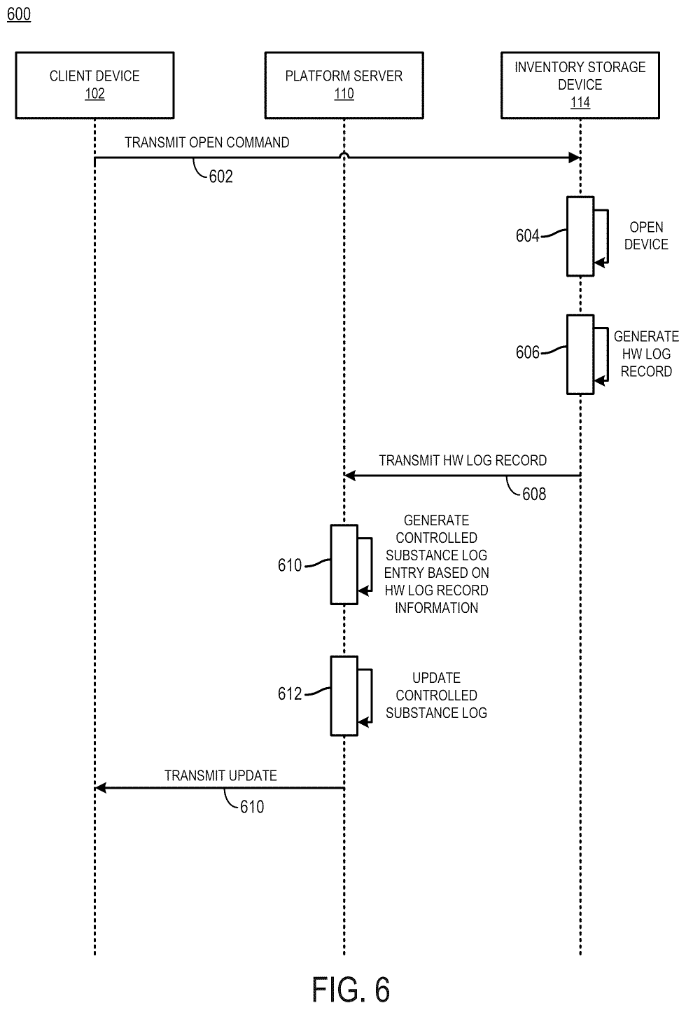

Referring now to FIG. 6, a sequence 600 between the client device 102, platform server 110, and inventory storage device 114 are shown. In 602, the client device 102 operated by a user within the medical practice transmits an “open” (or unlock) command to the inventory storage device 114. For example, the user may be in close proximity to the inventory storage device 114 with the inventory management application 104 in execution. The user may operate, through the user interface of the application 104, an open command, which transmits an open (unlock) command signal to the device 114. In response, the inventory storage device 114 may authenticate the user based on credentials submitted by the client device 104 and, in 604, cause the door 404 to open. Once opened, the inventory storage device 114 may transmit an “unlock” event signal to the application 104 and/or the platform application 112. In other embodiments, the inventory storage device 114, rather than receive a signal from the inventory management application 104, can also open the device based on user interaction with an access mechanism of the storage device 114 (e.g., based on a PIN authentication with a numpad on the device 114, using a card reader, biometric data, etc.). The inventory storage device 114 can log the access without receiving the information from the client device 104 and automatically generate a hardware log entry that can be used to create a controlled substance log entry.

In 606, the inventory storage device 114 may generate a hardware log record based on the user interaction with the user. The hardware log record may include information such as timestamp data, inventory item being removed (or stored), quantity, associated patient and/or client, doctor authorization, accessing user, and the like. In 608, the inventory storage device 114 transmit the hardware log record to the platform server 110. In turn, the platform server 110 generates a controlled substance log entry based on the information provided in the hardware log record. In 612, the platform server 110 updates the controlled substance log to include the generated entry. In 614, the platform server 110 transmits the update to the client device 102.

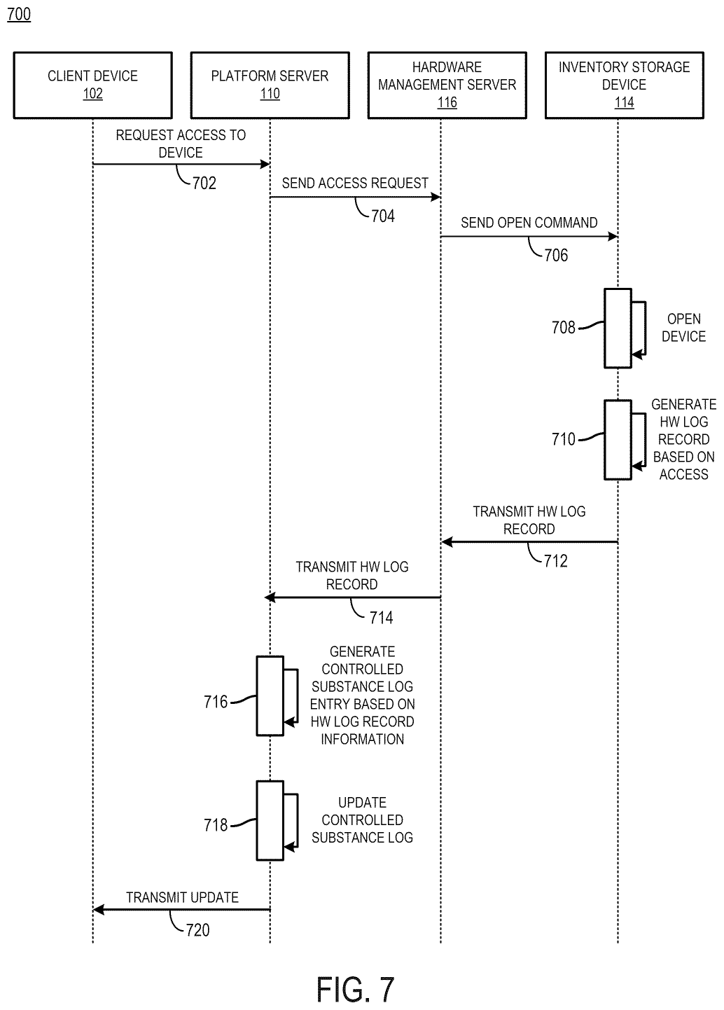

As stated, the system of the present disclosure may integrate with third-party storage lockers that can be managed by a third-party service, such as a hardware management server 116, to provide a hardware-agnostic approach for integration based on APIs exposed by both the platform server 110 and the hardware management server 116. Referring now to FIG. 7, a sequence 700 between the client device 102, platform server 110, hardware management server 116, and inventory storage device 114 are shown. In 702, the client device 102 requests access to the inventory storage device 114 and transmits the request to the platform server 110. The platform server 110 evaluates the request and, in 704, transmits the access request to the hardware management server 116. The hardware management server 116 may validate and authenticate the request and, in 706, transmit the “open” command to the inventory storage device 114 (e.g., by transmitting a signal causing the inventory storage device 114 to unlock its latch mechanism).

In response, the inventory storage device 114 opens in 708, enabling the user to access the inventory storage device 114. The inventory storage device 114 may, using sensors therein (e.g., camera sensors, weight sensors, positioning sensors, etc.), record the interaction between the user and the inventory storage device 114. In 710, the inventory storage device 114 generates a hardware log record based on the access. In 712, the inventory storage device 114 transmits the hardware log record to the hardware management server 116. In 714, the hardware management server 116 transmits the hardware log record to the platform server 110. In 716, the platform server 110 generates a controlled substance log entry based on the hardware log record information generated by the inventory storage server 114. In 718, the platform server 110 updates the controlled substance log based on the hardware log record. In 720, the platform server 110 transmits the update to the client device 102.

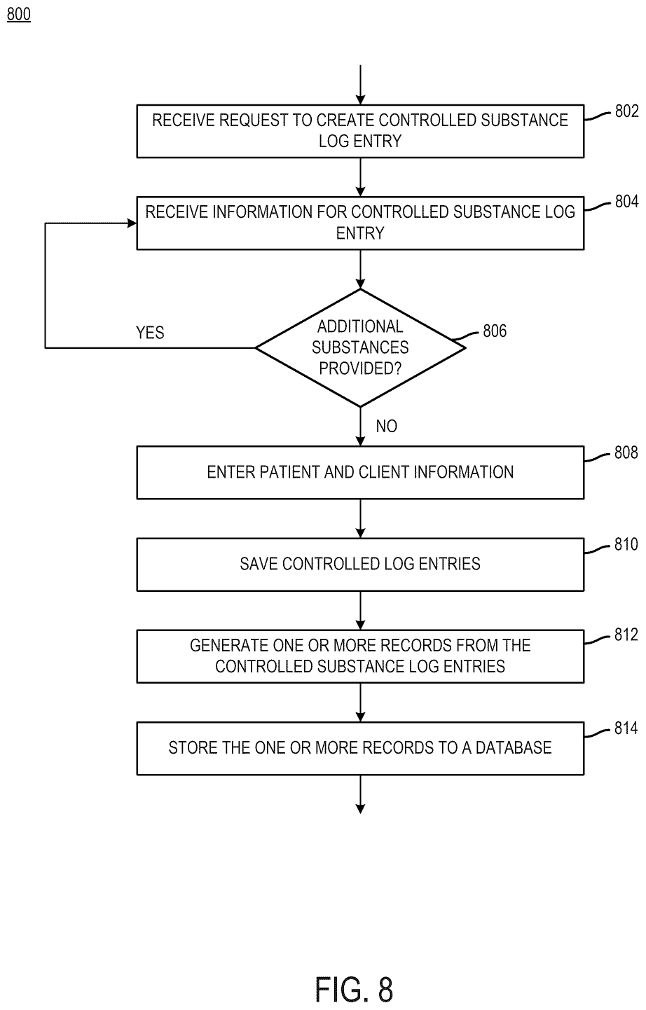

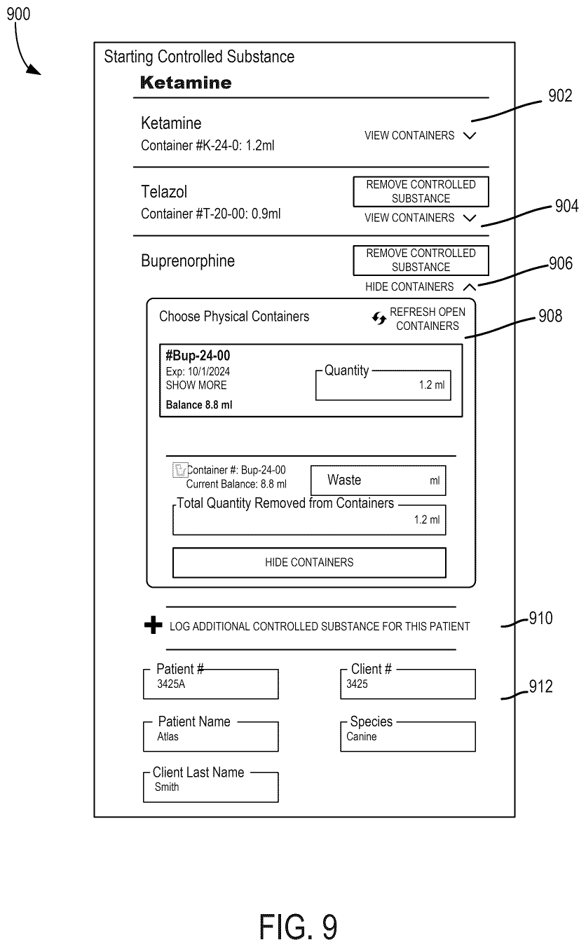

As stated, the inventory management system of the present disclosure is capable of generating multiple controlled substance log entries for a given patient or treatment from a single graphical interface view of the inventory management application 104. Referring now to FIG. 8, a method 800 for populating multiple controlled substance log entries for a given patient or treatment is shown. To provide additional clarity, FIG. 9 provides an example GUI view 900 of the inventory management application 104 for inputting multiple entries to the controlled substance log.

As shown, the method 800 begins in block 802, in which the inventory management application 104 receives a request to create a controlled substance log entry. For example, the inventory management application 104 may receive a command from a user from the GUI thereof to create an entry. As another example, the inventory management application 104 may be automatically triggered to do so in response to the user interacting with (or causing the inventory management application 104 to interact with) an inventory storage device 114. Particularly, when the user unlocks the hardware-based locker (e.g., via the inventory management application 104), the inventory management application 104 may generate a controlled substance log entry. From this entry, the user is able to create one or more controlled substance log record entries.

The user may select between different controlled substances in the GUI and select a starting controlled substance, as shown in portion 902 of FIG. 9. In an embodiment, the user is able to view by only one drug name at a time. From this view 900, the user can create a log entry for a specific controlled substance that is selected (e.g., via a click of a button on the graphical user interface).

In block 804, the inventory management application 104 receives information for the controlled substance log entry. The inventory management application 104 may prompt the user, via the user interface (e.g., using the view 900 of FIG. 9), for such information, which can include the drug name, specific container used, and a quantity. Portion 908 shows an example of such a prompt, in which the user has selected the drug Buprenorphine and clicked on the “View Containers” dropdown to access fillable fields. The user enters a drug quantity (drawn from the appropriate container), e.g., in a Log Entry view of the graphical user interface provided by the inventory management application 104. For example, assume Drug N represents the actual drug name that was selected in a given controlled substance log or hardware access log (e.g., if a user had Buprenorphine selected in the controlled substance log, the first drug that the user is entering the quantity for is Burenorphine).

In block 806, the inventory management application 104 determines whether additional substances are provided. If so, then the method 800 returns to block 804 for more drug inputs. Blocks 804 and 806 represent a continual loop enabling the user to enter quantities for other controlled substances into one or more logs for a given patient (e.g., the user can continue to include additional drugs by clicking on the Add icon in portion 910 for “Log Additional Controlled Substance for This Patient”). In block 808, once all desired controlled substance information is entered, the user may enter any remaining patient information associated with the controlled substance(s) (e.g., in the fields provided by portion 912 of the user interface view 900), which the inventory management application 104 receives. In block 810, the inventory management application 104 saves the entries provided within the user interface view 900 by the user. For example, the inventory management application 104 may save the entries to a temporary cache.

In block 812, the inventory management application 104 generates one or more records from the controlled substance log entries. For example, to do so, the inventory management application 104 may retrieve the entries from the cache and populate individual fields of a given controlled substance log entry with the values provided for a given entry by the user based on a mapping of fields between the controlled substance log and the hardware log. Previously entered information (e.g., drug name/type, quantities, patient information, client information, etc.) is split into one or more records based on how many controlled substances were added and N distinct controlled log records are created and saved in the database. For example, if three (3) drugs were added, the result would be three (3) distinct controlled substance log records in the database. This would be identical to the current result in which the user does not use any multilogging function and instead creates three separate records by going into each drug in the controlled substance log and entering data for each patient and client into the log, though less efficiently.

In block 814, the inventory management application 104 stores the one or more records to a database. Further, the inventory management application 104 may transmit the updated controlled substance log to the platform application 112. In turn, the platform application 112 may synchronize the controlled substance log stored thereon with the updated entries.

In some embodiments, all steps of the method 800 may be performed by the inventory management application 104. In other embodiments, the inventory management application 104 may be performed in part by the platform application 112 (e.g., generation of the multiple records from the user-provided entries, storing the records to the database, etc.).

Once complete, the inventory management application 104 may generate, from data provided by the user in the view, multiple controlled substance usage log records. Doing so advantageously reduces the amount of time taken to enter the records into the log.

While the foregoing is directed to embodiments of the present disclosure, other and further embodiments of the disclosure may be devised without departing from the basic scope thereof, and the scope thereof may be determined by the example claims that follow.

1. A method, comprising:
receiving, by a platform application executing on one or more processors, a request from a client device of a user to access an inventory storage device storing one or more controlled substances;
causing the inventory storage device to provide access to the client device;
receiving a hardware log generated by the inventory storage device, the hardware log providing one or more records relating to the access of the inventory storage device by the user, each record providing information relating to one of the one or more controlled substances removed from the inventory storage device;
generating, for each of the one or more records, an entry for a controlled substance log maintained by the platform application;
updating the controlled substance log with the generated entry for each of the one or more records.

2. The method of claim 1, wherein causing the inventory storage device to provide access to the client device comprises:
sending, via an application programming interface (API), a request to a hardware management server to unlock the inventory storage device, wherein, in response to receiving the request, the hardware management server transmits a signal to the inventory storage device.

3. The method of claim 2, further comprising, receiving the hardware log generated by the inventory storage device from the hardware management server.

4. The method of claim 1, wherein generating, for each of the one or more records, the entry for the controlled substance log comprises:
storing the one or more records in a cache;
populating fields of the entry with corresponding information from the one or more records in the cache based on a mapping between fields of the one or more records and the fields of the entry.

5. The method of claim 4, wherein the fields of the entry comprise a timestamp, controlled substance name, and a patient name.

6. The method of claim 1, further comprising, providing a graphical user interface (GUI) to the client device for adding a plurality of second controlled substance log entries, wherein the client device displays the GUI to prompt the user to provide information for the plurality of second controlled substance log entries via the GUI.

7. The method of claim 6, wherein the client device generates one or more of the plurality of second controlled substance log entries in response to the user providing information for the plurality of the second controlled substance log entries via the GUI.

8. A system comprising:
one or more processors; and
a memory storing a plurality of instructions, which, when executed on the one or more processors, causes the system to:
receive, by a platform application, a request from a client device of a user to access an inventory storage device storing one or more controlled substances;
cause the inventory storage device to provide access to the client device;
receive a hardware log generated by the inventory storage device, the hardware log providing one or more records relating to the access of the inventory storage device by the user, each record providing information relating to one of the one or more controlled substances removed from the inventory storage device;
generate, for each of the one or more records, an entry for a controlled substance log maintained by the platform application;
update the controlled substance log with the generated entry for each of the one or more records.

9. The system of claim 8, wherein to cause the inventory storage device to provide access to the client device comprises to send, via an application programming interface (API), a request to a hardware management server to unlock the inventory storage device, wherein, in response to receiving the request, the hardware management server transmits a signal to the inventory storage device.

10. The system of claim 9, wherein the plurality of instructions further causes the system to receive the hardware log generated by the inventory storage device from the hardware management server.

11. The system of claim 8, wherein to generate, for each of the one or more records, the entry for the controlled substance log comprises to:
store the one or more records in a cache;
populate fields of the entry with corresponding information from the one or more records in the cache based on a mapping between fields of the one or more records and the fields of the entry.

12. The system of claim 11, wherein the fields of the entry comprise a timestamp, controlled substance name, and a patient name.

13. The system of claim 8, wherein the plurality of instructions further causes the system to provide a graphical user interface (GUI) to the client device for adding a plurality of second controlled substance log entries, wherein the client device displays the GUI to prompt the user to provide information for the plurality of second controlled substance log entries via the GUI.

14. The system of claim 13, wherein the client device generates one or more of the plurality of second controlled substance log entries in response to the user providing information for the plurality of the second controlled substance log entries via the GUI.

15. One or more computer-readable storage media storing instructions, which, when executed on one or more processors, causes a system to:
receive, by a platform application executing on the one or more processors, a request from a client device of a user to access an inventory storage device storing one or more controlled substances;
cause the inventory storage device to provide access to the client device;
receive a hardware log generated by the inventory storage device, the hardware log providing one or more records relating to the access of the inventory storage device by the user, each record providing information relating to one of the one or more controlled substances removed from the inventory storage device;
generate, for each of the one or more records, an entry for a controlled substance log maintained by the platform application;
update the controlled substance log with the generated entry for each of the one or more records.

16. The one or more computer-readable storage media of claim 15, wherein to cause the inventory storage device to provide access to the client device comprises to send, via an application programming interface (API), a request to a hardware management server to unlock the inventory storage device, wherein, in response to receiving the request, the hardware management server transmits a signal to the inventory storage device.

17. The one or more computer-readable storage media of claim 16, wherein the plurality of instructions further causes the system to receive the hardware log generated by the inventory storage device from the hardware management server.

18. The one or more computer-readable storage media of claim 15, wherein to generate, for each of the one or more records, the entry for the controlled substance log comprises to:
store the one or more records in a cache;
populate fields of the entry with corresponding information from the one or more records in the cache based on a mapping between fields of the one or more records and the fields of the entry.

19. The one or more computer-readable storage media of claim 18, wherein the fields of the entry comprise a timestamp, controlled substance name, and a patient name.

20. The one or more computer-readable storage media of claim 15, wherein the plurality of instructions further causes the system to provide a graphical user interface (GUI) to the client device for adding a plurality of second controlled substance log entries, wherein the client device displays the GUI to prompt the user to provide information for the plurality of second controlled substance log entries via the GUI, wherein the client device generates one or more of the plurality of second controlled substance log entries in response to the user providing information for the plurality of the second controlled substance log entries via the GUI.
